(19) United States
(12) Patent Application Publication
(10) Pub. No.: US 2004/0143865 A1
(43) Pub. Date: Jul. 22, 2004

(54) Transgenic zebrafish models for angiogenesis

(76) Inventors: Amy L. Rubinstein, Atlanta, GA (US); Thanh Doan, Atlanta, GA (US); Shuo Lin, Los Angeles, CA (US)

Correspondence Address: NEEDLE & ROSENBERG, P.C., SUITE 1000, 999 PEACHTREE STREET, ATLANTA, GA, 30309-3915, US

(21) Appl. No.: 10/730,209

(22) Filed: Dec. 8, 2003

Related U.S. Application Data
(60) Provisional application No. 60/431,350, filed on Dec. 6, 2002.

(51) Int. Cl. A01K 67/027

(52) U.S. Cl. 800/20; 800/3

(57) ABSTRACT

The present invention relates to zebrafish models for the screening of compounds and genes that target angiogenesis in an in vivo model system.

This application claims priority to U.S. provisional application Serial No. 60/431,350 filed Dec. 6, 2002, which is herein incorporated by this reference in its entirety.

### FIELD OF THE INVENTION

The present invention relates to transgenic zebrafish models for the screening of compounds that target angiogenesis in an in vivo model system. The transgenic zebrafish angiogenesis models can also be used for target identification and validation.

### BACKGROUND

Angiogenesis is a process where blood vessels are formed from existing blood vessels. This involves proliferation, differentiation and migration of endothelial cells and possibly other cells found in the vasculature, such as smooth muscle cells and fibroblasts. Alteration of this process, either its potentiation or its inhibition, can be beneficial for the treatment of human diseases, such as cancer, macular degeneration, rheumatoid arthritis, Alzheimer's disease, wound healing, atherosclerosis and ischemia.

Inhibition of angiogenesis represents a powerful new approach to cancer therapy. To fully realize the potential of this avenue for cancer treatment, assays that can rapidly screen compounds for anti-angiogenic are necessary. Solid tumors require an adequate supply of blood vessels to survive, grow, and metastasize (Hanahan and Folkman, 1996; Li et al., 2000). New blood vessels that nourish growing tumors form by sprouting from existing blood vessels, a process known as angiogenesis. In recent years, angiogenesis has received considerable attention as a novel process to target for cancer drugs. Many drugs already in clinical trials have been shown to have anti-angiogenic activity and new drugs are being developed specifically for their ability to stop such blood vessel growth (Rosen, 2000). To date, anti-angiogenic drugs have had mixed success in clinical application. Many new compounds may need to be tested to identify drugs capable of treating a wide range of tumors. Thus, development of suitable assays for screening potential anti-angiogenic compounds is becoming increasingly important. The ideal assay should involve blood vessels growing in their natural environment, such as a whole living organism, yet be amenable to rapid analysis conducive to high throughput compound screening. To date, no current assays provide such a unique combination. The present invention provides an assay using the zebrafish (Danio rerio) that provides the relevance of an in vivo environment as well as the potential for high throughput drug screening.

The zebrafish has become a well-accepted model for studies of vertebrate developmental biology. Unlike the mouse, zebrafish embryos develop outside the mother and are transparent, facilitating the observation of differentiating tissues and organs. The vascular system, in particular, has been well described and shown to be highly conserved in the zebrafish (Isogai et al., 2001; Vogel and Weinstein, 2000). Furthermore, zebrafish embryos can live for several days without a significant blood supply, enabling one to study embryos with vascular defects. Many zebrafish blood vessels form by angiogenic sprouting and appear to require the same proteins shown to be necessary for blood vessel growth in mammals. In addition, the anti-angiogenic compound PTK787/ZK222584 has been shown to affect the formation of zebrafish blood vessels (Chan et al., 2002). Current methods of visualizing blood vessels in the zebrafish include whole mount in situ hybridization (Fouquet et al., 1997; Liao et al., 1997), detection of endogenous alkaline phosphatase activity and microangiography. The first two methods are time-consuming and involve fixation of embryos and larvae prior to analysis. Microangiography, a technique that involves injection of fluorescent beads into the circulation of living zebrafish larvae (Weinstein et al., 1996) is also labor-intensive and is only useful for visualization of patent blood vessels in a complete circulatory system. The present invention establishes a less labor-intensive way of visualizing blood vessels in the zebrafish, by generating a transgenic line of zebrafish that expresses a reporter protein, for example, green reef coral fluorescent protein (G-RCFP, Matz et al., 2000) or red fluorescent protein (dsRed2) specifically in blood vessels. It was found that the formation of intersegmental blood vessels, blocked by application of tyrosine kinase inhibitors that target the VEGF receptor, can be easily visualized in the fluorescent fish. Since no processing or microinjection is required, this assay is suitable for quantification in an automated assay. This transgenic fish line will greatly simplify the study of angiogenesis in the zebrafish, and therefore provide a valuable tool for screening compounds with anti-angiogenic potential.

The present invention provides a novel in vivo assay for anti-angiogenic and for pro-angiogenic compounds that utilizes transgenic zebrafish embryos with fluorescent blood vessels. To facilitate screening for angiogenesis inhibitors in a whole animal system, transgenic zebrafish lines that expresse the green reef coral fluorescent protein (G-RCFP) or red fluorescent protein (dsRed2) under the control of the blood vessel-specific VEGFR2 promoter were generated. The following invention demonstrates that tyrosine kinase inhibitors, targeting the vascular endothelial cell growth factor (VEGF) receptor, can effectively block new blood vessel formation in zebrafish embryos. In addition, recombinant proteins for VEGF can induce angiogenesis in transgenic zebrafish; therefore the transgenic zebrafish model may be used for detecting pro-angiogenic molecules. Furthermore, validation of gene targets for angiogenesis in the transgenic zebrafish was demonstrated using antisense molecules, such as morpholinos and gripNAs. All of these-processes can be easily visualized under a stereo microscope equipped with epifluorescence. Moreover, since zebrafish can produce hundreds of transparent embryos in each mating, this transgenic zebrafish line should be suitable for the development of an automated angiogenesis assay. Such an assay represents an improvement over current angiogenesis assays by combining the advantages of an in vivo context with a high throughput screening capability.

### SUMMARY OF THE INVENTION

The present invention provides a method of identifying an anti-angiogenic compound comprising: a) contacting a transgenic zebrafish that expresses a reporter protein in blood vessels, with a test compound; b) comparing the blood vessels in the zebrafish contacted with the test compound with the blood vessels of a transgenic zebrafish that was not contacted with the test compound and c) determining the effect of the test compound on blood vessel growth, such that if blood vessel growth in the zebrafish contacted with the test compound is less than blood vessel growth in the zebrafish that was not contacted with the test compound, the compound is an anti-angiogenic compound.

The present invention also provides a method of identifying a pro-angiogenic compound comprising: a) contacting a transgenic zebrafish that expresses a reporter protein in blood vessels, with a test compound; b) comparing the blood vessels in the zebrafish contacted with the test compound with the blood vessels of a transgenic zebrafish that was not contacted with the test compound and c) determining the effect of the test compound on blood vessel growth, such that if blood vessel growth in the zebrafish contacted with the test compound is greater than blood vessel growth in the zebrafish that was not contacted with the test compound, the compound is a pro-angiogenic compound.

The present invention further provides a method of identifying a blood vessel related gene that is involved in blood vessel growth comprising: a) comparing a transgenic zebrafish containing blood vessels that express a reporter protein, with a transgenic zebrafish containing blood vessels that express a reporter protein and has an altered blood vessel related gene; and b) determining the effect of the blood vessel related gene alteration on blood vessel growth such that if there is a difference between the blood vessels of the transgenic zebrafish containing blood vessels that express a reporter protein and the transgenic zebrafish containing blood vessels that express a reporter protein and has an altered blood vessel related gene, the blood vessel related gene is involved in blood vessel growth.

### BRIEF DESCRIPTION OF THE FIGURES

FIG. 1 shows the expression of G-RCFP and dsRed2 in zebrafish embryos, larvae and adults. (A-C), images were taken using a Leica stereo microscope equipped with epifluorescence. Scale bars=100 microns. G-RCFP expression mirrors that of endogenous expression at (A) 10 somites, side view, anterior to the left, arrows indicate clusters of cells in a bilateral stripe of VEGFR2:G-RCFP expressing cells; (B) 16 somites, dorsal view, anterior down, arrow shows where bilateral cells are merging in the midline; and (C) 24 hours, side view, anterior to the left. Arrow indicates an intersegmental vessel beginning to sprout from the underlying dorsal aorta. (D-G) 7 dpf larvae. Images were taken using a Zeiss laser scanning confocal microscope. G-RCFP is expressed in all blood vessels of the cranium and trunk. (D) Dorsal view, scale bar=100 microns. Example of an intersegmental vessel is indicated by the arrow. (E) Side view, scale bar=50 microns. Example of an intersegmental vessel is indicated by the arrow. (F) Ventral view, scale bar=100 microns. Arrow indicates blood vessels in the branchial arches. Subintestinal vessels are boxed; this region is enlarged in (G) Scale bar=25 microns. (H-J). Images were taken using a Leica stereo microscope equipped with epifluorescence (H-I) Adult TG(VEGFR2:G-RCFP) zebrafish. (H) head region. (I) Tail region. (J) TG(VEGFR2:dsRed2) zebrafish embryo at 72 hpf.

FIG. 2 shows zebrafish embryos, scale bar=100 microns. Yellow arrows indicate the dorsal aorta and red arrows indicate the caudal vein. Autofluorescent compounds concentrates in the yolk and yolk extension of treated embryos. (A) Untreated embryo at 30 hpf. An intersegmental vessel is indicated by the white arrow. (B) Embryo treated overnight with 10 μM SU5416 at 30 hpf. No intersegmental vessels are observed. (C) Embryo treated overnight with 15 μM SU6668 at 30 hpf. No intersegmental vessels are observed. (D) Larva at 5 dpf: treated overnight with 15 μM SU6668 at 2 dpf and allowed to recover for 3 days in fresh water. Intersegmental vessels are reforming. Examples of vessels that are migrating abnormally are indicated by the white arrows.

FIG. 3 shows disruption of pre-existing blood vessels in transgenic embryos. (A) Embryo at 24 hpf. Intersegmental vessels (example indicated by arrow) have begun to sprout from the underlying dorsal aorta. (B) Embryo at 30 hpf, treated with 10 μM SU5416 for six hours beginning at 24 hpf. A single intersegmental vessel (indicated by arrow) remains. (C) Untreated embryo at 30 hpf. Intersegmental vessels are intact (example indicated by arrow). Scale bar=100 microns.

FIG. 4 shows additional branching of fluorescent blood vessels following injection of human recombinant VEGF (hrVEGF). (A) Control TG(VEGFR2:G-RCFP) embryo injected with phenol red. Red arrow points to the basket of subintestinal vessels. (B) TG(VEGFR2:G-RCFP) embryo injected with hrVEGF. Red arrow points to additional branch off the subintestinal vessel basket.

FIG. 5 shows that antisense morpholinos can be used to show loss of gene function in transgenic zebrafish. (A) Control TG(VEGFR2:G-RCFP) at 24 hpf that was injected with phenol red at the 1-4 cell stage. (B) TG(VEGFR2:G-RCFP) at 24 hpf that was injected with a morpholino targeting G-RCFP at the 1-4 cell stage. (C) Graph showing the time course and dose dependence of the morpholino effect.

FIG. 6 shows that antisense GripNAs can be used to measure loss of gene function in transgenic zebrafish. (A) Control TG(VEGFR2:G-RCFP) at 48 hpf that was injected with phenol red at the 1-4 cell stage. (B) TG(VEGFR2:G-RCFP) at 48 hpf that was injected with a GripNA targeting G-RCFP at the 1-4 cell stage. (C) TG(VEGFR2:G-RCFP) at 48 hpf that was injected with a GripNA targeting G-RCFP at the 1-4 cell stage and again at 24 hpf with G-RCFP GripNA+Chariot II reagent.

FIG. 7 shows that an antisense morpholino directed against a known gene (VEGFR2) causes a predicted effect in TG(VEGFR2:G-RCFP) embryos. Arrows point to intersegmental blood vessels. (A) Control TG(VEGFR2:G-RCFP) embryo at 24 hpf that was injected with a phenol red at the 1-4 cell stage. (B) TG(VEGFR2:G-RCFP) embryo at 24 hpf that was injected with a morpholino targeting VEGFR2 at the 1-4 cell stage. Many intersegmental blood vessels are missing or shorter. (C) Control TG(VEGFR2:G-RCFP) embryo at 48 hpf that was injected with phenol red at the 1-4 cell stage. (D) TG(VEGFR2:G-RCFP) embryo at 48 hpf that was injected with a morpholino targeting VEGFR2 at the 1-4 cell stage. Some recovery of blood vessel formation has occurred, but intersegmental blood vessels are migrating in irregular patterns compared to the mock-injected (red arrow).

### DETAILED DESCRIPTION OF THE INVENTION

The present invention may be understood more readily by reference to the following detailed description of the preferred embodiments of the invention and the Example included therein.

Before the present compounds and methods are disclosed and described, it is to be understood that this invention is not limited to specific genes, specific proteins or specific methods. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise.

Identification of Anti-angiogenic and Pro-angiogenic Compounds

The present invention provides a method of identifying an anti-angiogenic compound comprising: a) contacting a transgenic zebrafish that expresses a reporter protein in blood vessels, with a test compound; b) comparing the blood vessels in the zebrafish contacted with the test compound with the blood vessels of a transgenic zebrafish that was not contacted with the test compound; and c) determining the effect of the test compound on blood vessel growth, such that if blood vessel growth in the zebrafish contacted with the test compound is less than blood vessel growth in the zebrafish that was not contacted with the test compound, the compound is an anti-angiogenic compound.

The transgenic zebrafish of this invention can be a transient or a stable transgenic zebrafish. The transgenic zebrafish in which the expression of a reporter protein is tissue-specific is contemplated for this invention. For example, transgenic animals that express a reporter protein at specific sites, such as blood vessels, can be produced by introducing a nucleic acid into fertilized eggs, embryonic stem cells or the germline of the animal, wherein the nucleic acid is under the control of a specific promoter which allows expression of the nucleic acid in specific types of cells (e.g., a promoter which allows expression only in blood vessels). As used herein, a protein or gene is expressed predominantly in a given tissue, cell type, cell lineage or cell, when 90% or greater of the observed expression occurs in the given tissue cell type, cell lineage or cell.

In the methods of the present invention, the control zebrafish, i.e. the zebrafish not contacted with the test compound can be an actual zebrafish that is being observed simultaneously with the zebrafish that is contacted with a test compound or the control zebrafish can be in the form of photographs or other information describing blood vessel growth in a zebrafish not contacted with a test compound. The skilled artisan can have a database of known blood vessel growth patterns and effects associated with particular stages of zebrafish blood vessel growth. This database can then be utilized to compare the blood vessel growth pattern of a zebrafish contacted with a test compound with the blood vessel growth patterns of untreated zebrafish at different stages of development.

As used herein, “blood vessel growth” refers to new blood vessel formation, growth of existing blood vessels and/or proliferation of blood vessels from an existing blood vessel(s),. Blood vessel growth can be quantified by measuring the change in fluorescence intensity, the number of fluorescent cells and/or the pattern of fluorescence in the transgenic zebrafish described herein. Blood vessel growth and changes in blood vessel growth can also be observed by visual inspection.

More specifically, this invention contemplates the use of a transgenic zebrafish that expresses a reporter protein that is under the control of a blood vessel specific expression sequence such as, but not limited to, the VEGFR2 promoter, the fli-1 promoter (Lawson and Weinstein, 2002), the tie-1 promoter and the tie-2 promoter.

The expression sequences used to drive expression of the reporter proteins can be isolated by one of skill in the art, for example, by screening a genomic zebrafish library for sequences upstream of the zebrafish gene of interest as described in the Examples. The expression sequences can include a promoter, an enhancer, a silencer and necessary information processing sites, such as ribosome binding sites, RNA splice sites, polyadenylation sites and transcriptional terminator sequences.

By utilizing a transgenic zebrafish that expresses a fluorescent protein under the control of a blood vessel specific promoter, the blood vessels can be visualized in the developing embryo and in later stages of zebrafish development. For example, one of skill in the art can monitor new blood vessel formation, growth of existing blood vessels and proliferation of blood vessels from existing blood vessels. The skilled artisan can also observe patterns of blood vessel formation and/or proliferation in the vasculature of the zebrafish. Thus, if a zebrafish of the present invention is exposed to an anti-angiogenic agent, its effects on blood vessels, i.e., reduction of blood vessel growth should be readily apparent by monitoring fluorescence. Zebrafish embryos can be easily “cultured” in 96 well plates where they can be soaked in test compounds. The effects of the test compound can also be readily visualized. If the test compound reduces blood vessel growth, one of skill in the art will be able to visualize this effect by observing the pattern of fluorescence in the zebrafish blood vessels. For example, if prior to administering a test compound, the skilled artisan observes fluorescent blood vessels and after administration of the test compound the total number of blood vessels is reduced or decreased growth of existing blood vessels is observed, the test compound reduces blood vessel growth and is thus, an anti-angiogenic compound. A decrease or reduction of blood vessel growth does not have to be complete as the efficacy of the test compound can range from a slight reduction in blood vessel growth to complete inhibition of blood vessel growth. This decrease or reduction can be a reduction in new blood vessel formation, a reduction in growth of existing blood vessels and/or a reduction in the proliferation of blood vessels from an existing blood vessel(s).

The transgenic fish utilized in the methods of this invention are produced by introducing a transgenic construct into cells of a zebrafish, preferably embryonic cells, and most preferably in a single cell embryo, essentially as described in Meng et al. (1998). The transgenic construct is preferably integrated into the genome of the zebrafish, however, the construct can also be constructed as an artificial chromosome. The transgenic construct can be introduced into embryonic cells using any technique known in the art. For example, microinjection, electroporation, liposomal delivery and particle gun bombardment can all be utilized to effect transgenic construct delivery to embryonic cells. Embryos can be microinjected at the one or two cell stage or the construct can be incorporated into embryonic stem cells which can later be incorporated into a growing embryo. Other methods for achieving zebrafish transgenesis that are developed can also be utilized to introduce a construct into an embryo or embryonic stem cells.

Embryos or embryonic cells can be obtained as described in the Examples provided herein. Zebrafish containing a transgene can be identified by numerous methods such as probing the genome of the zebrafish for the presence of the transgene construct by Northern or Southern blotting. Polymerase chain reaction techniques can also be employed to detect the presence of the transgene. Expression of the reporter protein can also be detected by methods known in the art. For example, RNA can be detected using any of numerous nucleic acid detection techniques. Alternatively, an antibody can be used to detect the expression product or one skilled in the art can visualize and quantify expression of a fluorescent reporter protein such as green reef coral fluorescent protein (G-RCFP), green fluorescent protein (GFP) and red fluorescent protein (dsRed2).

As used herein, a reporter protein is any protein that can be specifically detected when expressed. Reporter proteins are useful for detecting or quantifying expression from expression sequences. For example, operatively linking nucleotide sequences encoding a reporter protein to a tissue specific expression sequence allows one to study lineage development. In such studies, the reporter protein serves as a marker for monitoring developmental processes. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), green reef coral fluorescent protein (G-RCFP), cyan fluorescent protein (CFP), red fluorescent protein (RFP or dsRed2) and yellow fluorescent protein (YFP). For example, by utilizing GFP, fluorescence is observed upon exposure to ultraviolet light without the addition of a substrate. The use of reporter proteins that, like GFP, are directly detectable without requiring the addition of exogenous factors are preferred for detecting or assessing gene expression during zebrafish embryonic development. A transgenic zebrafish embryo, carrying a construct encoding a reporter protein and a tissue-specific expression sequence, such as an expression sequence that directs expression in blood vessels provides a rapid, real time in vivo system for analyzing spatial and temporal expression patterns of blood vessels and their interactions.

The test compounds used in the methods described herein can be made by methods standard in the art and include, but are not limited to, chemicals, small molecules, antisense molecules, siRNAs, drugs, antibodies, peptides and secreted proteins. Test compounds in the form of cDNAs can also be tested in the methods of the present invention. cDNAs can be injected into transgenic zebrafish embryos of the present invention in order to assess the effects of the polypeptides or proteins encoded by these cDNAs on blood vessel formation and/or growth.

In order to study the effects of anti-angiogenic agents on tumor growth, the present invention also contemplates methods in which tumor growth in the zebrafish of the present invention is promoted by the administration of chemicals, such as, 7,12-dimethylbenz[a]anthracene and ethylnitrosourea (Beckwith et al., 2000; Spitsbergen et al. 2000). Another method of inducing angiogenesis would be to grow the zebrafish embryos in hypoxic conditions—e.g., in a low oxygen and high nitrogen environment (Padilla and Roth, 2001).

The anti-angiogenic compound identified by the methods of the present invention can be utilized to treat disease states associated with angiogenesis or blood vessel growth. As used herein, “angiogenesis” is the process of blood vessel formation or blood vessel growth. When dysregulated, angiogenesis contributes to numerous malignant, inflammatory, infectious and immune disorders. Thus, the present invention contemplates treatment of such disorders with the anti-angiogenic compounds of the present invention. Angiogenesis, the natural process used by the human body to produce blood vessels, occurs as a pathological process in the development of solid tumors such as breast, colon, lung, pancreatic, prostate and brain cancers to name a few. The blood vessels created during this process provide the nutrients and oxygen the tumor needs to grow and spread. Therefore the anti-angiogenic compounds identified by the methods of the present invention can be utilized to treat cancerous tumors. Other disease states associated with angiogenesis include, but are not limited to rheumatoid arthritis, Alzheimer's disease and macular degeneration, which can also associated with overproduction of blood vessels. The anti-angiogenic compounds identified by the methods of the present invention can also be utilized in other in vitro assays to further evaluate the compound's effect on blood vessel growth and formation. Such in vitro assays are known in the art and can also be found in Jain, et al., 1997 and Auerbach, et al., 2000.

The present invention also provides methods for the identification of pro-angiogenic compounds. As utilized herein, a pro-angiogenic compound is a compound that promotes blood vessel growth. In other words, a pro-angiogenic compound is a compound that can be utilized to increase new blood vessel formation, increase growth of existing blood vessels and/or increased proliferation of blood vessels from an existing blood vessel(s).

Thus, the present invention provides a method of identifying a pro-angiogenic compound comprising: a) contacting a transgenic zebrafish that expresses a reporter protein in blood vessels, with a test compound; b) comparing the blood vessels in the zebrafish contacted with the test compound with the blood vessels of a transgenic zebrafish that was not contacted with the test compound and c) determining the effect of the test compound on blood vessel growth, such that if blood vessel growth in the zebrafish contacted with the test compound is greater than blood vessel growth in the zebrafish that was not contacted with the test compound, the compound is a pro-angiogenic compound. Those compounds found to promote or increase blood vessel growth can be utilized to treat diseases in which the promotion of blood vessel growth is desired. Such diseases include, but are not limited to, ischemia, atherosclerosis and wound healing.

Furthermore, the anti-angiogenic or pro-angiogenic compounds can be utilized in other in vivo animal models of angiogenesis or other disease states associated with angiogenesis, such as a mouse model, a rat model or a rabbit model of angiogenesis to study their therapeutic effects. For example, an anti-angiogenic compound identified by the methods of the present invention can be utilized in a mouse tumor model to assess its in vivo effects on tumor formation and progression. Similarly, a pro-angiogenic compound can be utilized in a mouse or rat model of ischemia to assess its in vivo effects.

Further provided by the present invention is a method of making an anti-angiogenic compound comprising: a) synthesizing a test compound; b)contacting a transgenic zebrafish that expresses a reporter protein in blood vessels, with a test compound;c) comparing the blood vessels in the zebrafish contacted with the test compound with the blood vessels of a transgenic zebrafish that was not contacted with the test compound; and d) determining the effect of the test compound on blood vessel growth, such that if blood vessel growth in the zebrafish contacted with the test compound is less than blood vessel growth in the zebrafish that was not contacted with the test compound, a compound with anti-angiogenic activity was made.

Also provided by the present invention is a method of making a pro-angiogenic compound comprising: a) synthesizing a test compound; b) contacting a transgenic zebrafish that expresses a reporter protein in blood vessels, with a test compound; c) comparing the blood vessels in the zebrafish contacted with the test compound with the blood vessels of a transgenic zebrafish that was not contacted with the test compound; d) determining the effect of the test compound on blood vessel growth, such that if blood vessel growth in the zebrafish contacted with the test compound is greater than blood vessel growth in the zebrafish that was not contacted with the test compound, a compound with pro-angiogenic activity was made.

One of skill in the art will know that the compounds of the present invention can be administered to a subject in a suitably acceptable pharmaceutical carrier. The subject can be any mammal, preferably human, and can include, but is not limited to mouse, rat, cow, guinea pig, hamster, rabbit, cat, dog, goat, sheep, monkey, horse and chimpanzee. By pharmaceutically acceptable is meant a material that is not biologically or otherwise undesirable, i.e., the material may be administered to an individual along with the selected agent without causing any undesirable biological effects or interacting in a deleterious manner with any of the other components of the pharmaceutical composition in which it is contained. In addition, one can include other medicinal agents, pharmaceutical agents, carriers, adjuvants, diluents, etc.

The compounds of the present invention can be administrated via intratumoral injection, oral administration, nebulization, inhalation, mucosal administration, intranasal administration, intratracheal administration, intravenous administration, intraperitoneal administration, subcutaneous administration and intramuscular administration.

Dosages of the compositions of the present invention will also depend upon the type and/or severity of the disease and the individual subject's status (e.g., species, weight, disease state, etc.) Dosages will also depend upon the form of the composition being administered and the mode of administration. Such dosages are known in the art or can be determined by one of skill in the art.

Furthermore, the dosage can be adjusted according to the typical dosage for the specific disease or condition to be treated. Often a single dose can be sufficient; however, the dose can be repeated if desirable. The dosage should not be so large as to cause adverse side effects. Generally, the dosage will vary with the age, condition, sex and other parameters and can be determined by one of skill in the art according to routine methods (see e.g., Remington's Pharmaceutical Sciences). The individual physician in the event of any complication can also adjust the dosage.

Identification of Blood Vessel Related Genes

Also provided by the present invention is a method of identifying blood vessel related genes comprising: a) constructing a zebrafish blood vessel cDNA library; and b) identifying a blood vessel related gene. Construction of the library is accomplished as described in the Examples and as known by those of skill in the art.

As utilized herein, “a blood vessel related gene” is a gene that is associated with the growth and/or development of blood vessels. This gene can be a blood vessel specific gene, involved primarily in the growth and/or development of blood vessels, or a gene that is associated with blood vessel growth and/or development as well as other cellular or physiological processes. Upon identification of blood vessel related genes, one of skill in the art would know how to compare the zebrafish sequence with other sequences in available databases in order to identify a human homologue of a blood vessel related zebrafish gene. One of skill in the art would also be able to identify other homologues such as a mouse homologue or a rat homologue. Alternatively, sequences from the blood vessel related zebrafish gene can be utilized as probes to screen a human library and identify human homologs. The zebrafish sequences can also be utilized to screen other animal libraries, such as a mouse library or a rat library. Upon identification of a mouse, rat or other animal homologue, these sequences can be utilized to screen for a human homologue, either by searching available databases, or screening a human library.

Upon identification of a blood vessel related gene, the present invention also contemplates altering blood vessel related genes in zebrafish in order to determine their role in blood vessel formation, growth and function.

For example, a transgenic zebrafish of the present invention that expresses a reporter protein in blood vessels can also have a blood vessel related gene altered. One of skill in the art would compare embryonic development of this altered fish with a non-altered transgenic zebrafish expressing a reporter protein in blood vessels. If there is a difference in the characteristics of the blood vessels and their interactions (i.e. blood vessel formation, growth, maturation and proliferation), the gene that has been altered plays a role in normal blood vessel development. Gene alteration in zebrafish can be accomplished by, but is not limited to, a mutation, a deletion, an insertion, the use of antisense molecule (such as morpholinos and gripNAs), overexpression of RNA or cDNA, targeted genetic knockout, random genetic knockout and any other alteration that results in the alteration of a blood vessel related gene. The alterations contemplated by the present invention can result in a decrease or an increase in expression of the blood vessel related gene product.

Thus, the present invention also provides a method of identifying a blood vessel related gene that is involved in blood vessel growth comprising: a) comparing the blood vessels of a transgenic zebrafish that expresses a reporter protein, with the blood vessels of a transgenic zebrafish that expresses a reporter protein and has an altered blood vessel related gene; and b) determining the effect of the altered blood vessel related gene on blood vessels such that if there is a difference between the blood vessels of the transgenic zebrafish that express a reporter protein and the blood vessels of the transgenic zebrafish that express a reporter protein and has an altered blood vessel related gene, the blood vessel related gene is involved in blood vessel growth.

Target Identification and Validation

Also provided by the present invention is a method of identifying a blood vessel related gene as a target for an anti-angiogenic compound comprising: a) contacting a transgenic zebrafish containing blood vessels that express a reporter protein with an anti-angiogenic compound; b) contacting a transgenic zebrafish containing blood vessels that express a reporter protein and has an altered blood vessel related gene with an anti-angiogenic compound; c) comparing the transgenic zebrafish containing blood vessels that express a reporter protein with the transgenic zebrafish containing blood vessels that express a reporter protein and has an altered blood related gene; and d) determining the effect of the anti-angiogenic compound on blood vessel growth, such that if blood vessel growth is different in the transgenic zebrafish with blood vessels that express a reporter protein as compared to the zebrafish containing blood vessels that express a reporter protein and has an altered blood vessel related gene, the blood vessel related gene is a candidate target for the anti-angiogenic compound.

Also provided by the present invention is a method of identifying a blood vessel related gene as a target for a pro-angiogenic compound comprising: a) contacting a transgenic zebrafish containing blood vessels that express a reporter protein with a pro-angiogenic compound; b) contacting a transgenic zebrafish containing blood vessels that express a reporter protein and has an altered blood vessel related gene with a pro-angiogenic compound; c) comparing the transgenic zebrafish containing blood vessels that express a reporter protein with the transgenic zebrafish containing blood vessels that express a reporter protein and has an altered blood vessel related gene; and d) determining the effect of the pro-angiogenic compound on blood vessel growth, such that if there is a change in blood vessel growth in the transgenic zebrafish with blood vessels that expresses a reporter protein as compared to the zebrafish containing blood vessels that expresses a reporter protein and has an altered blood vessel related gene, the blood vessel related gene is a potential target for the pro-angiogenic compound.

The present invention also provides a method of identifying a pro-angiogenic blood vessel related gene that modulates the effects of an anti-angiogenic compound comprising: a) contacting a transgenic zebrafish containing blood vessels that express a reporter protein with an anti-angiogenic compound; b) contacting a transgenic zebrafish containing blood vessels that express a reporter protein and has an altered blood vessel related gene with an anti-angiogenic compound; c) comparing the transgenic zebrafish containing blood vessels that express a reporter protein with the transgenic zebrafish containing blood vessels that express a reporter protein and has an altered blood vessel related gene; and d) determining the effect of the altered gene on blood vessel growth, such that if blood vessel growth in the transgenic zebrafish containing blood vessels that express a reporter protein is less than blood vessel growth in the zebrafish containing blood vessels that express a reporter protein and has an altered blood vessel related gene, the blood vessel related gene is a pro-angiogenic gene that modulates the effects of an anti-angiogenic compound.

Further provided by the present invention is a method of identifying an anti-angiogenic blood vessel related gene that modulates the effects of a pro-angiogenic compound comprising: a) contacting a transgenic zebrafish containing blood vessels that express a reporter protein with a pro-angiogenic compound; b) contacting a transgenic zebrafish containing blood vessels that express a reporter protein and has an altered blood vessel related gene with a pro-angiogenic compound; c) comparing the transgenic zebrafish containing blood vessels that express a reporter protein with the transgenic zebrafish containing blood vessels that express a reporter protein and has an altered blood vessel related gene; and d) determining the effect of the altered gene on blood vessel growth, such that if blood vessel growth in the transgenic zebrafish containing blood vessels that express a reporter protein is greater than blood vessel growth in the zebrafish containing blood vessels that express a reporter protein and has an altered blood vessel related gene, the blood vessel related gene is an anti-angiogenic gene that modulates the effects of a pro-angiogenic compound.

The zebrafish containing an altered blood vessel related gene can also be utilized in the method of the present invention in order to identify potential drug targets. These genetically altered zebrafish can be utilized in the methods described herein to assess the effects of anti-angiogenic or pro-angiogenic compounds.

Therefore, the present invention also provides a method of identifying an anti-angiogenic compound that affects blood vessel growth via a blood vessel related gene comprising: a) contacting a transgenic zebrafish containing blood vessels that express a reporter protein with a test compound; b) contacting a transgenic zebrafish containing blood vessels that express a reporter protein and has an altered blood vessel related gene with a test compound; c) comparing the transgenic zebrafish containing blood vessels that express a reporter protein with the transgenic zebrafish containing blood vessels that express a reporter protein and has an altered blood vessel related gene; and d) determining the effect of the test compound on blood vessel growth, such that if blood vessel growth in the transgenic zebrafish containing blood vessels that express a reporter protein is less than blood vessel growth in the zebrafish containing blood vessels that express a reporter protein and has an altered blood vessel related gene, the compound is an anti-angiogenic compound that affects blood vessel growth via the blood vessel related gene that has been altered.

The present invention also provides a method of identifying a pro-angiogenic compound that affects blood vessel growth via a blood vessel related gene comprising: a) contacting a transgenic zebrafish containing blood vessels that express a reporter protein with a test compound; b) contacting a transgenic zebrafish containing blood vessels that express a reporter protein and has an altered blood vessel related gene with a test compound; c) comparing the transgenic zebrafish containing blood vessels that express a reporter protein with the transgenic zebrafish containing blood vessels that express a reporter protein and has an altered blood vessel related gene; and d) determining the effect of the test compound on blood vessel growth, such that if there is an increase in blood vessel growth in the transgenic zebrafish containing blood vessels that express a reporter protein as compared to blood vessel growth in the zebrafish containing blood vessels that express a reporter protein and has an altered blood vessel related gene, the compound is a pro-angiogenic compound that affects blood vessel growth via the blood vessel related gene that has been altered.

Also provided by the present invention is a method of identifying a pro-angiogenic compound that modulates the effects of an anti-angiogenic blood vessel related gene: a) contacting a transgenic zebrafish containing blood vessels that express a reporter protein and has an altered anti-angiogenic blood vessel related gene with a test compound; b) comparing the transgenic zebrafish containing blood vessels that express a reporter protein and has an altered blood vessel-related gene contacted with the test compound with the blood vessels of a transgenic zebrafish containing blood vessels that express a reporter protein and has an altered blood vessel-related gene that was not contacted with the test compound; and c) determining the effect of the test compound on blood vessel growth, such that if blood vessel growth in the transgenic zebrafish containing blood vessels that express a reporter protein and has an altered blood vessel related gene contacted with a test compound is greater than blood vessel growth in the zebrafish containing blood vessels that express a reporter protein and has an altered blood vessel related gene, the test compound is a pro-angiogenic compound that modulates the effects of an anti-angiogenic blood vessel related gene.

The present invention also provides a method of identifying an anti-angiogenic compound that modulates the effects of a pro-angiogenic blood vessel related gene: a) contacting a transgenic zebrafish containing blood vessels that express a reporter protein and has an altered pro-angiogenic blood vessel related gene with a test compound; b) comparing the transgenic zebrafish containing blood vessels that express a reporter protein and has an altered blood vessel-related gene contacted with the test compound with the blood vessels of a transgenic zebrafish containing blood vessels that express a reporter protein and has an altered blood vessel-related gene that was not contacted with the test compound; and c) determining the effect of the test compound on blood vessel growth, such that if blood vessel growth in the transgenic zebrafish containing blood vessels that express a reporter protein and has an altered blood vessel related gene contacted with a test compound is less than blood vessel growth in the zebrafish containing blood vessels that express a reporter protein and has an altered blood vessel related gene, the test compound is an anti-angiogenic compound that modulates the effects of a pro-angiogenic blood vessel related gene.

In the methods described above, either fish can receive the test compound first. One of skill in the art would then compare the genetically altered zebrafish expressing the reporter protein with the zebrafish expressing a reporter protein in blood vessels that does not have a blood vessel related gene altered. It is also possible for one of skill in the art to contact only the genetically altered zebrafish with a test compound and compare it to known patterns of blood vessel formation and/or blood vessel growth associated with the non-altered zebrafish expressing a reporter protein in blood vessels. If a decrease in blood vessel growth is observed in the zebrafish expressing a reporter protein in blood vessels, that does not have a blood vessel related gene altered as compared to the genetically altered zebrafish, the test compound is an anti-angiogenic agent that affects blood vessel growth via the blood vessel related gene that has been altered. The anti-angiogenic agent may be interfering with transcription of this gene, translation of a protein encoded by the blood vessel related gene or it may be inhibiting the blood vessel related protein's activity either by inhibiting its ability to interact with other proteins, or degrading the protein.

The present invention is more particularly described in the following examples which are intended as illustrative only since numerous modifications and variations therein will be apparent to those skilled in the art.

### EXAMPLES

Zebrafish. The Tubingen wild-type strain was used to make the transgenic line, described below. Fish were maintained at 28° C. in a recirculating aquaculture system, using methods described in the Zebrafish Book (Westerfield, 1995).. An animal care protocol for these experiments was approved by the Institutional Animal Care and Use Committee (IACUC) at Georgia State University, Atlanta, Ga.

Transgenic fish production. A zebrafish BAC library (Incyte) was screened by the polymerase chain reaction (PCR) for genomic clones containing the VEGFR2 gene (forward primer: 5′ TTTCTCCATTCGTCTTAGA 3′, reverse primer: 5′ CTCCGTATGTCACTTCACGT 3′; PCR conditions: 94° C., 1 minute; for 35 cycles: 94° C., 15 seconds, 62° C., 1 minute, 72° C., 90 seconds; 72° C., 10 minutes). A 10 kb BamH1-EcoR1 fragment of the VEGFR2 positive BAC clone containing the 5′ end of the VEGFR2 gene was cloned into pBluescript (Stratagene). The polymerase chain reaction (PCR) was used to amplify a 6.5 kb piece using the T7 primer from pBluescript and a primer designed upstream from the VEGFR2 start codon (5′ CTACACTATGTAGTGAAGGTG 3′; PCR conditions: 94° C., 1 minute; 35 cycles of 94° C., 15 seconds, 60° C., 1 minute, 72° C., 5 minutes; 72° C., 7 minutes). This 6.5 kb 5′ flanking sequence, described as the promoter region for VEGFR2, was cloned into a vector containing the G-RCFP gene. A linear fragment that contains the 5′ VEGFR2 flanking sequence or promoter region, G-RCFP gene and the SV40 polyadenylation signal was injected into embryos at the one cell stage. Embryos that exhibited mosaic transient expression of G-RCFP in blood vessels were raised to adulthood. These fish were screened to identify founders (F₀) that carried the VEGFR2:G-RCFP transgene. A founder fish was mated to wild-type fish and their fluorescent offspring were raised to form the F₁ generation. F₁ fish were mated to each other to create homozygous stocks.

Other reporter systems in addition to G-RCFP can be employed. Transgenic zebrafish expressing dsRed2 specifically in blood vessels were also created. Once the VEGFR2:G-RCFP construct was made, the coding region for G-RCFP was cloned out of the vector and replaced with coding sequence for dsRed2. The linearized VEGFR2:dsRed2 construct was injected into embryos at the one cell stage and later screened for founders (F₀). The transgenic offsprings (F₁)were raised and crossed to create homozygous offspring.

Compound preparation. 100 mM stocks of SU5416 and SU6668 were prepared by dissolving dry compounds in 100% DMSO. These stocks were then diluted in sterile fish water and aliquoted into 24 well plates prior to embryo treatments. Due to the light-sensitive nature of the compounds, plates were wrapped in aluminum foil for angiogenesis experiments.

Angiogenesis experiments. For the experiments described herein, embryos from matings between heterozygous F₁ fish and wild-type partners were used. 50% of embryos from these crosses were fluorescent and were sorted at the 10-somite stage. Fluorescent embryos were manually dechorionated and arrayed into 24 well plates before being subjected to anti-angiogenic compounds at the 13-somite stage. For some experiments, embryos were treated starting at the beginning of gastrulation (shield stage, 6 hpf), or allowed to develop until 24 hpf before application of compounds. For those embryos analyzed after one day of development, phenylthiourea can be added to the water to suppress pigmentation. Fish and embryos were maintained at 28° C.

Transgenic fish construction. VEGFR2 (previously referred to as Flk-1 or KDR) is one of several receptors for vascular endothelial cell growth factor (VEGF) family members in humans and is expressed specifically in blood vessels (Gale and Yancopolous, 1999). It is required for the formation of endothelial cells and blood vessels during embryogenesis, The VEGFR2 gene is one of the earliest known markers of the hemangioblast lineage and its expression is exquisitely tissue-specific. Therefore, the promoter of the zebrafish homologue of VEGFR2 was isolated to create a transgenic zebrafish line that would express a fluorescent protein specifically in blood vessels.

A partial zebrafish VEGFR2 gene had been cloned previously and its expression pattern described (Fouquet et al., 1997; Liao et al., 1997; Thompson et al., 1998). More recently, a full-length VEGFR2 cDNA was described (Chan et al., 2002). Based on these published sequences, a 6.5 kb genomic fragment 5′to the VEGFR2 initiation codon that drives G-RCFP or dsRed2 expression specifically in zebrafish blood vessels was isolated. Hereafter, these transgenic lines are referred to as TG(VEGFR2:G-RCFP) or TG(VEGFR2:dsRed2), respectively, using standard zebrafish nomenclature.

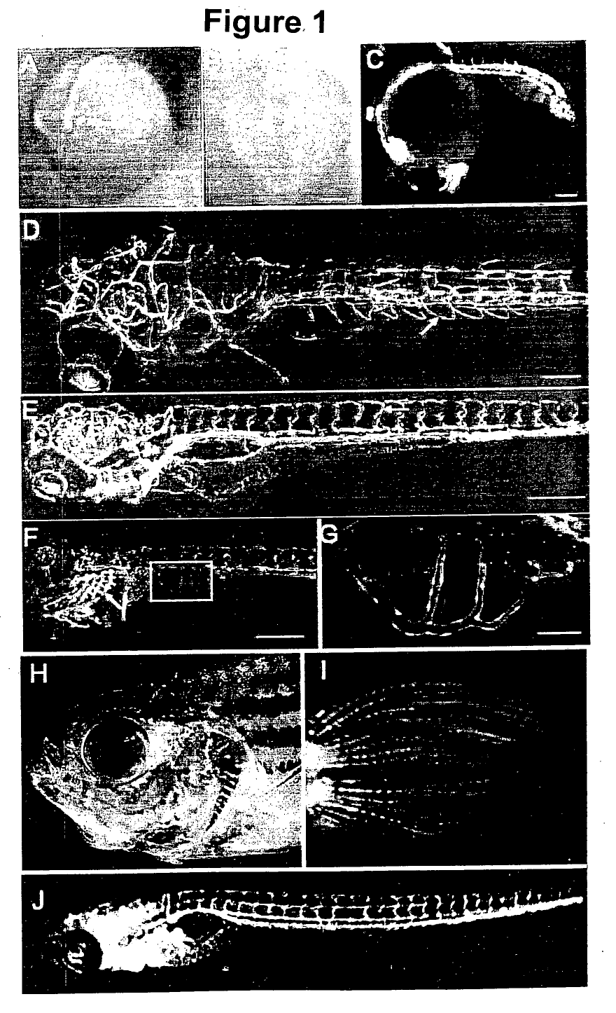

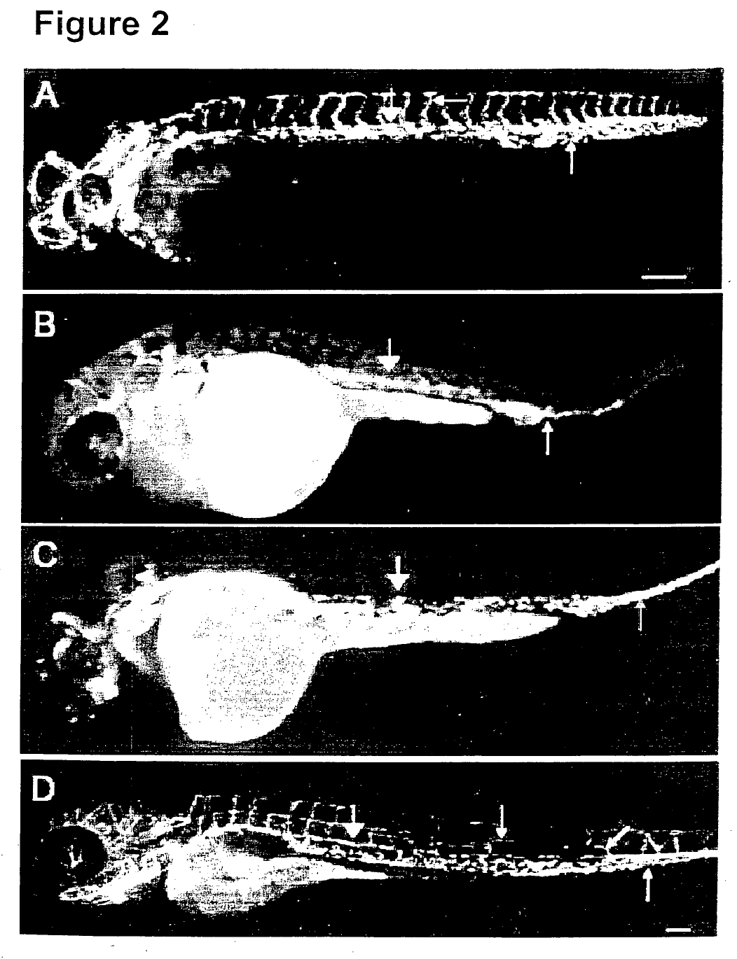

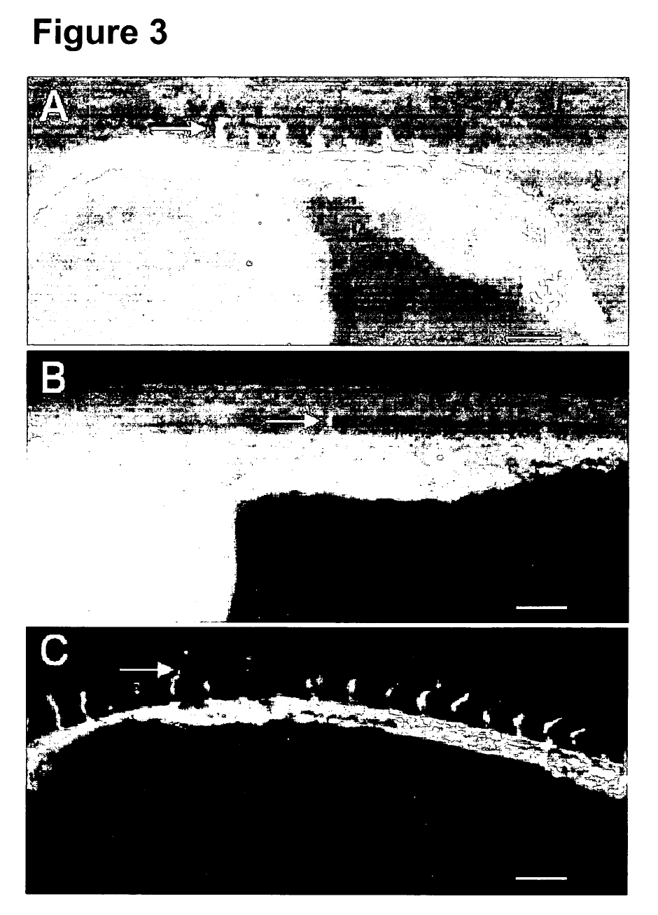

Stable transgenic zebrafish embryos begin to express G-RCFP at about the 10-somite stage, in bilateral stripes extending from around the eyes to a region lateral to developing somites forming in the trunk, as shown in FIG. 1A. Fluorescent protein expression extends both rostrally and caudally over the next few hours of development. The bilateral VEGFR2-expressing cells begin to migrate to the midline (FIG. 2A), where they fuse to form the dorsal aorta by 24 hours post fertilization (hpf). FIG. 3A shows intersegmental vessels, which begin to sprout from the underlying dorsal aorta at around 24 hpf. These expression patterns are consistent with the endogenous VEGFR2 gene expression, determined by whole-mount in situ hybridization (Fouquet et al., 1997; Liao et al., 1997). By 7 days post fertilization (dpf), G-RCFP is observed in all blood vessels, including the dorsal aorta, caudal artery, caudal veins, intersegmental vessels, cranial vessels and subintestinal vessels, as shown in FIG. 1D-G. The expression of G-RCFP in zebrafish persisted through adulthood (FIG. 1H-I). A similar spatial and temporal expression pattern was observed for TG(VEGFR2:dsRed2) zebrafish (FIG. 1J).

The bright fluorescence observed in the blood vessels of these transgenic embryos make them ideal for the development of an in vivo assay for angiogenesis inhibitors. Another transgenic line of zebrafish with fluorescent blood vessels has been described previously, which utilized a mouse tie-2 promoter to drive expression of GFP in blood vessels (Motoike et al., 2000). By comparison, the blood vessels in the VEGFR2 transgenic line of this invention are significantly brighter. In addition, fluorescence is detected at an earlier stage of development (10-somite stage compared to 12-somite stage), and lasts longer, being clearly visible in all blood vessels as late as 10 dpf. The fluorescence can also be observed in adult zebrafish, particularly in the gills, abdominal region, dorsal fins and caudal fins. Recently, a second transgenic line was described that expresses enhanced green fluorescent protein (eGFP) under the control of the promoter for the fli-1 gene, which encodes a transcription factor expressed early in the hematopoietic cell lineage and later in blood vessels (Lawson and Weinstein, 2002). eGFP expression was also detected in cranial neural crest in this line. Therefore, the TG(VEGFR2:G-RCFP) line of this invention exhibits more specific expression of fluorescent protein in blood vessels, making it more valuable for high throughput compound screening.

Isolation of fluorescent blood vessel cells. Embryos produced by the mating of transgenic males and females are dechorionated in pronase solution, washed and allowed to develop in 28° C. until isolation of cells. Embryos are then disrupted in Holtfreter's solution (60 mM NaCl, 2.4 mM sodium bicarbonate, 0.8 mM calcium chloride, 0.67 mM potassium chloride) using a 1.5 ml pellet pestle (Kontes Glass, OEM749521-1590). After digestion with 1×Trypsin/EDTA for 15 minutes at 32° C., the cells are washed twice with phosphate buffered saline (PBS) and passed through a 40 micron nylon mesh filter. Cells are recovered by centrifugation at 400×g for 5 minutes in a Beckman tabletop centrifuge. Fluoresence activated cell-sorting (FACS) is performed using a standard protocol for isolating fluorescein-labeled cells. For the final sorting, fluorescent cells are sorted directly into a buffer containing guanidinium isothiocyanate and stored at −70° C. until use.

RNA isolation. Total RNA is extracted from FACS-purified cells using the Trizol RNA Isolation Kit (LIFE TECHNOLOGIES, Grand Island, N.Y.) and MRNA is isolated from the total RNA using PolyATtract System 1000 (Promega, Madison, Wis.). The protocols provided by LIFE TECHNOLOGIES and Promega are utilized for isolation of MRNA. At least 50 ng of mRNA will be prepared for cDNA library construction.

cDNA library construction. The SMART cDNA Library Construction Kit (Clontech), which was developed for constructing high-quality cDNA libraries from small quantities of RNA is utilized. As discussed above, although either total or poly A+ RNA may be used as a template for SMART cDNA synthesis, mRNA is utilized for the purposes of the present invention.

First-Strand cDNA is synthesized using 25 ng polyA+ MRNA isolated from GFP-positive cells. SMART/5′ oligonucleotide III and CDS/3′ oligonucleotide III is used in the MMLV reverse transcriptase reaction. The SMART/5′ oligonucleotide III contains an Sfi I site with AAT whereas the CDS/3′ oligonucleotide III contains an Sfi I site with GGC. This variation of AAT and GGC is used because Sfi I recognizes 5′GGCCNNNNNGGCC3′.

Low cycle, long-distance PCR (LD-PCR) is used to amplify the first-strand cDNA. KlenTaq Polymerase, a new 5′ PCR primer complementary to the SMART/5′ oligonucleotide III, and the CDS/3′ oligonucleotide III are used in the reaction. Currently, it is possible to amplify enough PCR products for library construction after 10 cycles. After amplification, a sample of the PCR product is analyzed with 1-kb DNA ladder size markers to determine the size and amount of PCR product.

As mentioned above, SMART oligonucleotide III and CDS oligonucleotide III contain Sfi I restriction sites. PCR products are digested with Sfi I restriction enzyme. This digestion generates DNA fragments with 5′ AAT and 3′ GGC overhangs. Digested products are then size-fractionated. Two cDNA pools are collected: one is 1 -2kb and another one is larger than 2kb. After purification, the size-fractionated, Sfi I-digested cDNA is ligated to the dephosphorylated and Sfi I digested lambda TriplEx vector. One of these arms has a Sfi I site with TTA whereas the other one has a SfiI with CCG. Therefore, the cDNA inserts are cloned into the phage arms with their 5′ ends at the TTA arms and the 3′ ends at the CCG arms. The ligated products are packaged and a small portion of it plated out on LB plates for titering. 1-2×10⁶ independent clones are usually obtained. If the titer is as expected, remaining phages are converted into plasmid, to simplify sequencing and subtraction, as described below.

Library characterization. First, 1,000 random clones from the library are sequenced. This provides insight into the quality of the library, including the level of redundancy. Plasmid DNA obtained from the first 1,000 clones are used as driver to subtract redundant clones from the rest of the library. Normalization and subtraction is done according to Bonaldo, et al. (Bonaldo, et al., 1996). Clones are sequenced until it is decided that all potential expressed sequences from the platelet library have been identified.

The identification of blood vessel related genes from a library would also be known to one of skill in the art Tissue specificity of genes identified from the blood vessel cDNA library will be determined by in situ hybridization as described by Thisse, et al. (1993). Antisense RNA probes will be synthesized by in vitro transcription, incorporating digoxygenin-labeled UTP (Roche Molecular Biochemicals). Embryos will be hybridized with probe overnight at 70 degrees Celsius in 50% formamide buffer. After several washes, embryos will be incubated overnight with an antibody to digoxygenin conjugated to alkaline phosphatase (Roche Molecular Biochemicals). After several additional washes, embryos will be developed in an alkaline solution containing nitro blue tetrazolium (NBT) and 5-bromo-4-chloro-3-indolyl phosphate (BCIP).

To determine whether a particular blood-vessel related gene is important for angiogenesis (and thus may be a useful drug target), the following can be done: Functional information can be obtained by knockdown technology, such as morpholinos (Nasevicius and Ekker, 2000) or gripNAs (Urtishak et al., 2003). Transient over-expression and over-expression of dominant negative constructs, when appropriate, is also used to provide functional information.

Assay development. The bright, consistent fluorescence of angiogenic blood vessels in TG(VEGFR2:G-RCFP) transgenic zebrafish embryos show that they could provide an ideal tool for testing angiogenesis drugs. Jain, et al. (1997) describe several factors that are important for the design of an optimal angiogenesis assay, including ease of experimentation, cost-effectiveness, rapidity, reproducibility, and ability to quantify vessel formation. Adult zebrafish produce thousands of eggs routinely and are relatively inexpensive to maintain, thus fulfilling the first two criteria.

To test the utility of the TG(VEGFR2:G-RCFP) transgenic line for angiogenesis drug screening, embryos were subjected to compounds known to have anti-angiogenic activity, including SU5416 (Fong et al., 1999; Mendel et al., 2000a) and SU6668 (Laird et al., 2000), two indolinone-based small molecules demonstrated to inhibit VEGF-induced vascular endothelial cell proliferation in vitro. Furthermore, SU5416 and SU6668 have been reported to inhibit the formation of metastases and microvessel formation and increase apoptosis of both tumor cells and tumor endothelial cells in mouse xenograft models (Shaheen et al., 1999). SU5416 is believed to specifically target VEGF receptors (Fong et al., 1999; Mendel et al., 2000a), while SU6668 inhibits the basic fibroblast growth factor receptor and the platelet-derived growth factor receptor in addition to the VEGF receptors(Laird et al., 2000).

Zebrafish embryos were initially incubated with SU5416 and SU6668 overnight, beginning when embryos were at the 13-somite stage (approximately 15 hpf). While formation of the primary vasculature has begun by this stage, angiogenic sprouting of intersegmental and head vessels does not begin until much later in development. Thus, this stage represents a reasonable starting point for application of compounds. Following overnight treatment in compounds, embryos were rinsed extensively and soaked in fresh water for two to four hours. Rinsing was found to improve the ability to assay fluorescence in the blood vessels, since both compounds are autofluorescent.

As shown in FIG. 2A-C, overnight application of both compounds completely blocked intersegmental vessel formation, while preserving fluorescence in the dorsal aorta, caudal artery, caudal veins, and major cranial vessels. In addition, blood vessels in the head that form by angiogenesis were not observed in treated embryos. Treated embryos also exhibited an enlarged pericardial cavity and evidence of blood pooling in the ventral tail. These findings are reminiscent of the phenotype observed following knockout of VEGF-A in the zebrafish by antisense morpholino (Nasevicius et al., 2000), suggesting that these effects result from lack of a functioning vascular system rather than non-specific toxicity. The concentrations of the compounds required to see these effects (10 μM for SU5416 and 15 μM for SU6668) are similar to the plasma level of SU5416 (5 μM) in patients treated in clinical trials (Rosen, 2000; Mendel et al., 2000b). When embryos were treated at an even earlier stage (6 hpf), before any blood vessel formation has begun, the results were not significantly different. Data from these experiments is summarized in Table I.

Since it is known that intersegmental vessels form in part through cell migration (Childs et al., 2002), it is possible that SU5416 and SU6668 have a more general effect on cell migration in treated embryos, rather than a specific effect on angiogenesis. Therefore, treated embryos were examined at the 18 somite stage, after bilateral VEGFR2 expressing cells have begun to migrate toward the midline. No difference was observed in the migration pattern of VEGFR2 expressing cells between control and drug-treated embryos, suggesting that these compounds do not affect endothelial cell migration or the overall patterning of the vasculature.

To determine whether angiogenesis could resume after the embryos are removed from the compounds, the embryos were allowed to recover in fresh water for 24-72 hours following application of compounds. It was found that blood vessels do begin to sprout again from the dorsal aorta, but they often appear irregular in shape and position, when compared to untreated embryos, as shown in FIG. 2D. It is known that intersegmental blood vessel patterning is dependent upon signals originating from the somites (Childs et al., 2002). While the somites in drug-treated embryos appeared normal, it is possible that molecules required for correct patterning of intersegmental vessels are not present at this later stage of development. Alternatively, drug treatment may affect expression of chemoattractant and/or repulsive signals in the somites.

As expected, the embryos treated with SU5416 required a longer recovery time than those treated with SU6668, suggesting that its effects are longer lasting. A similar finding has been observed when cultured human vascular endothelial cells are treated with these compounds. This has been explained by the fact that SU5416 is highly lipophilic and more likely to remain sequestered in cell membranes after excess compound has been washed out (Mendel et al., 2000b). This explanation is likely to apply to zebrafish as well, since it has been noticed by this laboratory that SU5416 is highly concentrated in the yolk of treated embryos and is difficult to remove even after many hours of soaking in fresh water.

To determine whether SU5416 and SU6668 can alter blood vessel formation at later stages of development, embryos were treated at 24 hpf, when intersegmental vessels have begun sprouting. It was found that a six-hour treatment disrupted pre- existing blood vessels, as shown in FIG. 3 and Table I. This finding shows that endothelial cells that are already part of existing blood vessels can be damaged by these VEGF receptor inhibitors. Furthermore, such breakdown of specific cell types can be clearly visualized by the disappearance of fluorescent protein in these transgenic embryos. However, this effect appears to be limited to newly forming blood vessels, since the well-established primary vasculature is not affected by compound application. It has previously been demonstrated that both SU5416 and SU6668 affect the survival of endothelial cells within tumors, by increasing the level of apoptosis in these cells (Shaheen et al., 1999). Thus, a similar mechanism may be responsible for the destruction of newly formed blood vessels in zebrafish embryos.

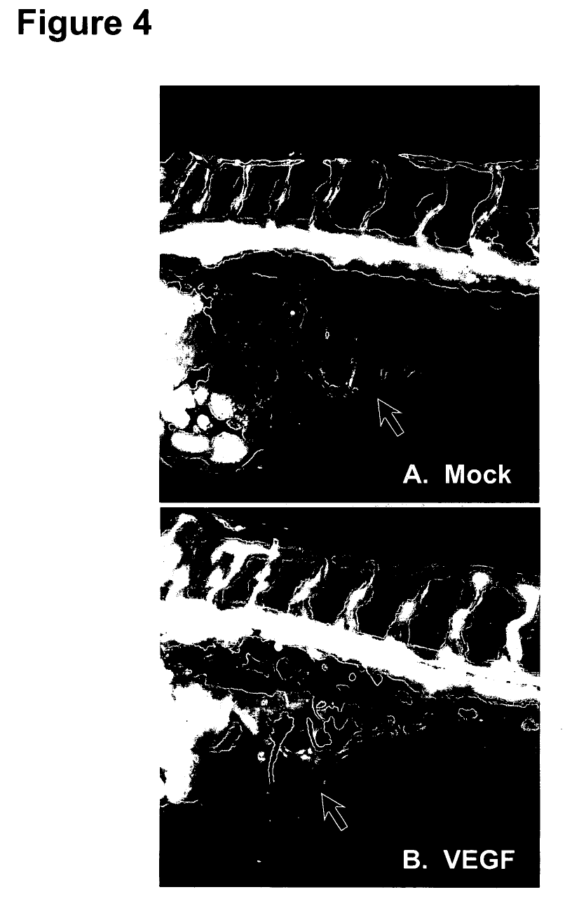

To assess whether pro-angiogenesis can be examined in TG(VEGFR2:G-RCFP) zebrafish, human recombinant VEGF (hrVEGF) was injected into the heart cavity of transgenic zebrafish embryos at 48 hpf and fluorescence observed at 72 hpf (FIG. 4). hrVEGF resulted in an increase in the amount of fluorescence in existing blood vessels, such as intersegmental vessels and dorsal aorta. In addition, hrVEGF induced additional branching of subintestinal blood vessels, as shown in FIG. 4. The increase in fluorescence of existing blood vessels appeared to be due to an increase in cell proliferation, as suggested by an increase in the number of nuclei within the blood vessels. Taken together, this data shows that the transgenic zebrafish can be used to track pro-angiogenic effects and therefore, can be used to discover compounds that induce angiogenesis.

The TG(VEGFR2:G-RCFP) transgenic fish can form the basis of an in vivo assay for angiogenesis inhibitors and agonists. Zebrafish embryos can be easily arrayed in 96 well plates and subjected to a large number of different compounds. This approach has been used successfully to identify compounds that interfere with early development in the zebrafish (Peterson et al., 2000). The dramatic changes in G-RCFP fluorescence that were observed following application of angiogenesis inhibitors or agonists should be easily quantifiable and with new developments in microplate reader capabilities, it is possible to develop a system for rapidly screening thousands of molecules per week for anti-angiogenic and pro-angiogenic activity. Many problems are associated with current in vivo angiogenesis assays. For example, some are expensive and labor intensive and others are difficult to quantify (Auerbach et al., 2000). One of the most popular assays, the mouse corneal angiogenesis assay, is performed using a tissue that is normally avascular, raising questions about the relevance of this assay to in vivo angiogenesis (Auerbach et al., 2000). Given the importance of this area for the identification of new drugs for treatment of cancer and other diseases, a simple, potentially quantitative, relatively inexpensive assay, such as that described herein, would be a major addition to the field of angiogenesis drug screening.

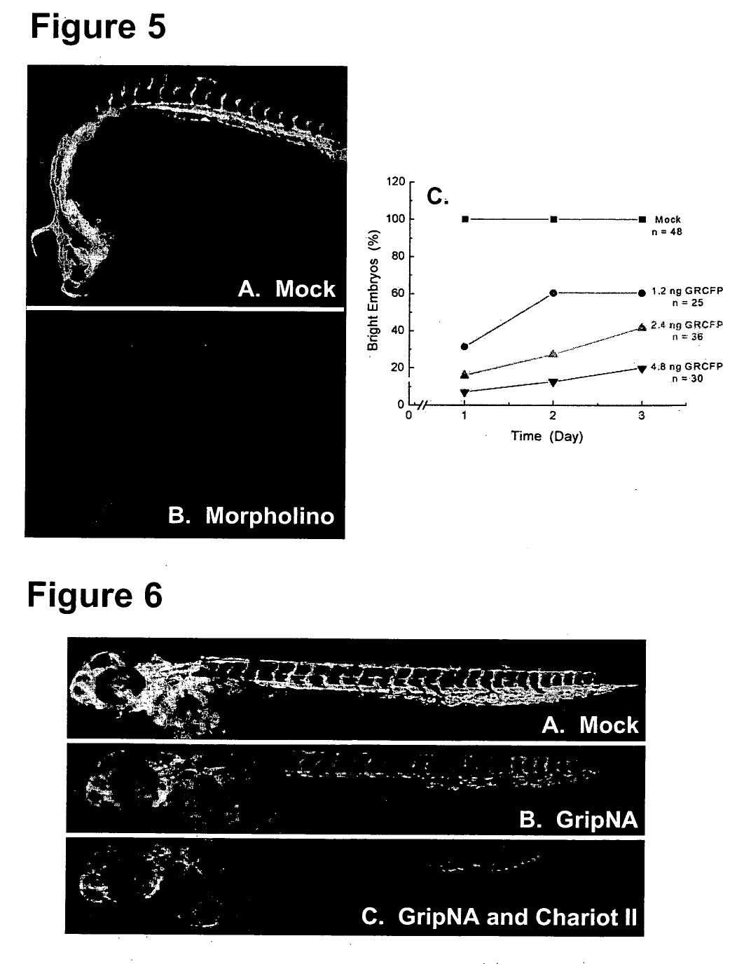

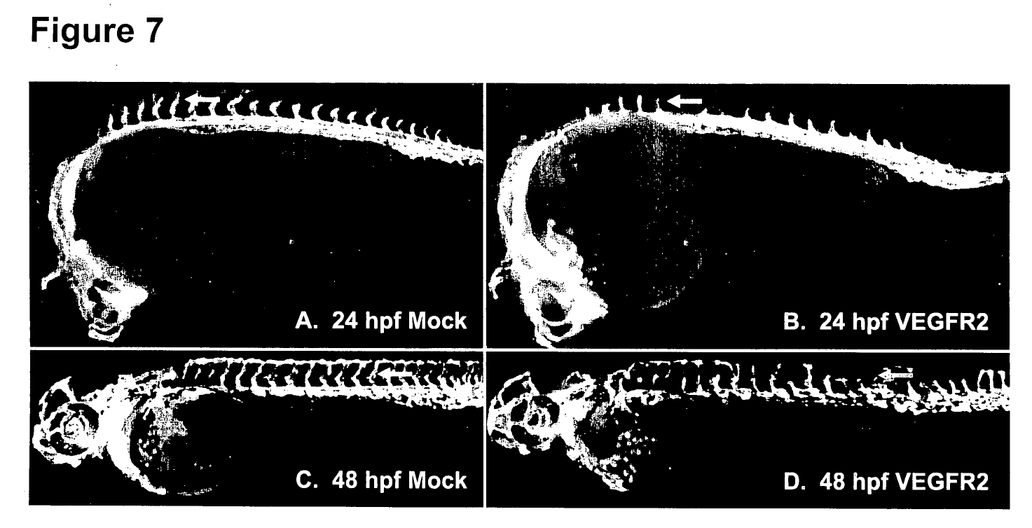

Transgenic zebrafish with fluorescent blood vessels can also be used for validation of genetic targets that may have roles in either promotion of or inhibition of blood vessel formation.; This can be achieved by using antisense molecules, such as morpholinos and gripNAs, to knockdown the function of a specific gene or group of genes. For example, antisense morpholino recognizing G-RCFP (FIG. 5) or antisense gripNA recognizing G-RCFP (FIG. 6) were injected into TG(VEGFR2:G-RCFP) embryos at the one to four cell stage. At 24 hpf, the expression of G-RCFP was significantly reduced in the morpholino-injected embryos when compared to mock-injected (0.2% phenol red) embryos (FIG. 5). The antisense morpholino produced a time-dependent and dose-dependent effect, as shown on the graph in FIG. 5. Antisense gripNA recognizing G-RCFP was also effective in zebrafish. When embryos were injected at 1-4 cell stage with or without gripNA, a reduction in fluorescence was observed (FIG. 6).

The delivery and use of the antisense molecule was not limited to the 1-4 cell stage. When the gripNA-treated embryos were further injected with Chariot II (Active Motif) and antisense gripNA recognizing G-RCFP at 24 dpf and then observed at 48 hpf, a reduction in fluorescence of the transgenic zebrafish was observed when compared to mock-injected embryos or embryos injected only with gripNA at the 1-4 cell stage. This shows that delivery and effectiveness of the antisense molecule can be accomplished at later stages of development of zebrafish (FIG. 6). Taken together, this data shows that both types of antisense molecules, morpholino and gripNA, can be effective in reducing protein expression in transgenic zebrafish. To further explore the application of antisense molecules for angiogenesis, antisense morpholinos targeting the VEGFR2 gene were injected into TG(VEGFR2:G-RCFP) embryos at the 1-4 cell stage. VEGFR2 is known to be required for normal development of blood vessels in both the mouse and the zebrafish (Shalaby et al., 1995; Habeck et al., 2002). VEGFR2-morpholino-injected embryos exhibited reduced fluorescent blood vessel development at 24 hpf. By 48 hpf, intersegmental blood vessels began to regrow, but in an irregular pattern when compared to mock-injected (0.2% phenol red) embryos (FIG. 7). Thus, the TG(VEGFR2:G-RCFP) zebrafish can be used to validate the function of a gene involved in angiogenesis.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### References

Auerbach, R., Akhtar, N., Lewis, R. L. & Shinners, B. L.(2000). Angiogenesis assays: problems and pitfalls. Canc. Metast. Rev. 19, 167-172.

Beckwith L G, Moore J L, Tsao-Wu G S, Harshbarger J C, Cheng K C. (2000). Ethylnitrosourea induces neoplasia in zebrafish (Danio rerio). Lab Invest. 80, 379-85.

Bonaldo M F, Lennon G, Soares M B. (1996). Normalization and subtraction: two approaches to facilitate gene discovery. Genome Res. 6: 791-806

Chan, J., Bayliss, P. E., Wood, J. M. & Roberts, T. M. (2002). Dissection of angiogenic signaling in zebrafish using a chemical genetic approach. Cancer Cell 1, 257-267.

Childs, S., Chen, J-N., Garrity, D. M. & Fishman, M. C. (2002). Patterning of angiogenesis in the zebrafish embryo. Development 129, 973-982.

Fong, T. A. et al. (1999). SU5416 is a potent and selective inhibitor of the vascular endothelial growth factor receptor (Flk-1/KDR) that inhibits tyrosine kinase catalysis, tumor vascularization, and growth of multiple tumor types. Cancer Res. 59, 99-106.

Fouquet, B., Weinstein, B. M., Serluca, F. C. & Fishman, M. C. (1997). Vessel patterning in the embryo of the zebrafish: guidance by notochord. Dev. Biol. 183, 37-48.

Gale, N. W. & Yancopoulos, G. D. (1999). Growth factors acting via endothelial cell-specific receptor tyrosine kinases: VEGFs, angiopoietins, and ephrins in vascular development. Genes Dev. 13, 1055-1066.

Habeck H, Odenthal J, Walderich B, Maischein H, Schulte-Merker S; Tubingen 2000 screen consortium. (2002). Analysis of a zebrafish VEGF receptor mutant reveals specific disruption of angiogenesis. Curr Biol. 12, 1405-1412.

Hanahan, D. & Folkman, J. (1996). Patterns and emerging mechanisms of the angiogenic switch during tumorigenesis. Cell 86, 353-364.

Isogai, S., Horiguchi, M. & Weinstein, B. M. (2001). The vascular anatomy of the developing zebrafish: an atlas of embryonic and early larval development. Dev. Biol. 230, 278-301.

Jain, R. K., Schlenger, K., Hockel, M. & Yuan, F. (1997). Quantitative angiogenesis assays: Progress and problems. Nat. Med. 3, 1203-1208.

Laird, A. D. et al.(2000). SU6668 is a potent anti-angiogenic and antitumor agent that induces regression of established tumors. Cancer Res. 60, 4152-4160.

Lawson, N. D. & Weinstein, B. M. (2002). In vivo imaging of embryonic vascular development using transgenic zebrafish. Dev. Biol. 248, 307-318.

Li, C. Y., Shan, S., Cao, Y. & Dewhirst, M. W. (2000). Role of incipient angiogenesis in cancer metastasis. Cancer Met. Rev. 19, 7-11.

Liao, W. et al. The zebrafish gene cloche acts upstream of a flk-1 homologue to regulate endothelial cell differentiation. Development 124, 381-389 (1997).

Matz, M. V. et al. (2000). Fluorescent proteins from nonbioluminescent Anthozoa species. Nat. Biotech. 17, 969-973.

Mendel D B, et al. (2000a). Development of SU5416, a selective small molecule inhibitor of VEGF receptor tyrosine kinase activity, as an anti-angiogenesis agent. Anti-Cancer Drug Des. 15, 29-41.

Mendel, D. B. et al. (2000b). The angiogenesis inhibitor SU5416 has long-lasting effects on vascular endothelial growth factor receptor phosphorylation and function. Clin. Canc. Res. 6, 4848-4858.

Meng, A M., Jessen, J H. and Lin, S. (1998). Transgenesis. Methods in Cell Biology: Zebrafish. Edited by Detrich, W., Zon, L I and Westerfield, M. Academic Press, pp. 133-148.

Motoike, T. et al. (2000). Universal GFP reporter for the study of vascular development. Genesis 28, 75-81.

Nasevicius A, & Ekker S C. (2000). Effective targeted gene ‘knockdown’ in zebrafish. Nat Genet. 26, 216-20.

Nasevicius, A., Larson, J. & Ekker, S C. (2000). Distinct requirements for zebrafish angiogenesis revealed by a VEGF-A morphant. Yeast 17, 294-301.

Padilla P A and Roth M B. (2001). Oxygen deprivation causes suspended animation in the zebrafish embryo. Proc. Natl. Acad. Sci. USA 98, 7331-7335.

Peterson, R. T, Link, B. A., Dowling, J. E. & Schreiber, S. L. (2000). Small molecule developmental screens reveal the logic and timing of vertebrate development. Proc. Natl. Acad. Sci. USA 97, 12965-12969.

Rosen, L. (2000). Anti-angiogenic strategies and agents in clinical trials. Oncologist 5(suppl. 1), 20-27.

Shaheen R. M. et al. (1999). Anti-angiogenic therapy targeting the tyrosine kinase receptor for vascular endothelial growth factor receptor inhibits the growth of colon cancer liver metastasis and induces tumor and endothelial cell apoptosis. Cancer Res. 59, 5412-5416.

Shalaby F, Rossant J, Yamaguchi T P, Gertsenstein M, Wu XF, Breitman M L, Schuh A C. (1995). Failure of blood-island formation and vasculogenesis in Flk-1-deficient mice. Nature. 376, 62-66.

Spitsbergen J M, Tsai H W, Reddy A, Miller T, Arbogast D, Hendricks J D, Bailey G S. (2000). Neoplasia in zebrafish (Danio rerio) treated with 7,12-dimethylbenz[a]anthracene by two exposure routes at different developmental stages. Toxicol Pathol. 28, 705-15.

Thisse C, Thisse B, Schilling T F, Postlethwait J H. (1993). Structure of the zebrafish snaill gene and its expression in wild-type, spadetail and no tail mutant embryos. Development 119, 1203-1215.

Thompson, M. A. et al. (1998). The cloche and spadetail genes differentially affect hematopoiesis and vasculogenesis. Dev. Biol. 197, 248-269.

Urtishak K A, Choob M, Tian X, Sternheim N, Talbot W S, Wickstrom E, Farber S A. (2003). Targeted gene knockdown in zebrafish using negatively charged peptide nucleic acid mimics. DevDyn. 228, 405-413.

Vogel, A. M. & Weinstein, B. M. (2000). Studying vascular development in the zebrafish. Trends Cardiovasc. Med. 10, 352-360.

Weinstein, B. M., Stemple, D. L., Driever, W. & Fishman, M. C. (1996). Gridlock, a localized heritable vascular patterning defect in the zebrafish. Nat. Med. 1, 1143-1147.

Westerfield M. The Zebrafish Book (University of Oregon Press, Eugene, Oreg., 1995).

TABLE I

| | Stage at | | | Percent | |
|---|---|---|---|---|---|
| | treat- | Time | | full | Number |
| | ment | Treated | Phenotypic response | effect | treated |
| SU5416 | | | | | |
| 5 μM | 6 hpf | 24 h | Dorsal aorta, caudal | 100% | 50 |
| | 15 hpf | 24 h | veins, present. | 77% | 136 |
| | 24 hpf | 6 h | Intersegmental vessels | 60% | 45 |
| | | | absent. | | |
| 10 μM | 6 hpf | 24 h | Cranial vessels reduced | 100% | 50 |
| | 15 hpf | 24 h | in number. | 100% | 136 |
| | 15 hpf | 6 h | | 100% | 45 |
| | 24 hpf | 6 h | | 100% | 120 |
| SU6668 | | | | | |
| 10 μM | 6 hpf | 24 h | Dorsal aorta, caudal | 100% | 50 |
| | 15 hpf | 24 h | veins, present. | 100% | 136 |
| | 24 hpf | 6 h | Intersegmental vessels | 87% | 45 |
| | | | absent. | | |
| 15 μM | 6 hpf | 24 h | Cranial vessels reduced | 100% | 50 |
| | 15 hpf | 24 h | in number. | 100% | 136 |
| | 15 hpf | 6 h | | 100% | 45 |
| | 24 hpf | 6 h | | 100% | 120 |

What is claimed is:

1. A method of identifying an anti-angiogenic compound comprising:
a) contacting a transgenic zebrafish that expresses a reporter protein in blood vessels, with a test compound;
b) comparing the blood vessels in the zebrafish contacted with the test compound with the blood vessels of a transgenic zebrafish that was not contacted with the test compound.
c) determining the effect of the test compound on blood vessel growth, such that if blood vessel growth in the zebrafish contacted with the test compound is less than blood vessel growth in the zebrafish that was not contacted with the test compound, the compound is an anti-angiogenic compound.

2. The method of claim 1, wherein the reporter protein is a fluorescent protein.

3. The method of claim 2, wherein the fluorescent protein is green reef coral fluorescent protein.

4. The method of claim 2, wherein the fluorescent protein is a red fluorescent protein.

5. The method of claim 1, wherein the reporter protein is under the control of a VEGFR2 expression sequence.

6. A method of identifying a pro-angiogenic compound comprising:
a) contacting a transgenic zebrafish that expresses a reporter protein in blood vessels, with a test compound;
b) comparing the blood vessels in the zebrafish contacted with the test compound with the blood vessels of a transgenic zebrafish that was not contacted with the test compound.
c) determining the effect of the test compound on blood vessel growth, such that if blood vessel growth in the zebrafish contacted with the test compound is greater than blood vessel growth in the zebrafish that was not contacted with the test compound, the compound is a pro-angiogenic compound.

7. The method of claim 6, wherein the reporter protein is a fluorescent protein.

8. The method of claim 7, wherein the fluorescent protein is green reef coral fluorescent protein.

9. The method of claim 7, wherein the fluorescent protein is a red fluorescent protein.

10. The method of claim 6, wherein the reporter protein is under the control of a VEGFR2 expression sequence.

11. A method of identifying a blood vessel related gene that is involved in blood vessel growth comprising:
a) comparing a transgenic zebrafish containing blood vessels that express a reporter protein, with a transgenic zebrafish containing blood vessels that express a reporter protein and has an altered blood vessel related gene; and
b) determining the effect of the altered blood vessel related gene on blood vessel growth such that if there is a difference between the blood vessels of the transgenic zebrafish containing blood vessels that express a reporter protein and the transgenic zebrafish containing blood vessels that express a reporter protein and has an altered blood vessel related gene, the blood vessel related gene is involved in blood vessel growth.

12. The method of claim 11, wherein the reporter protein is a fluorescent protein.

13. The method of claim 12, wherein the fluorescent protein is green reef coral fluorescent protein.

14. The method of claim 12, wherein the fluorescent protein is a red fluorescent protein.

15. The method of claim 11, wherein the reporter protein is under the control of a VEGFR2 expression sequence.

16. A method of identifying a pro-angiogenic blood vessel related gene that modulates the effects of an anti-angiogenic compound comprising:
a) contacting a transgenic zebrafish containing blood vessels that express a reporter protein with an anti-angiogenic compound;
b) contacting a transgenic zebrafish containing blood vessels that express a reporter protein and has an altered blood vessel related gene with an anti-angiogenic compound;
c) comparing the transgenic zebrafish containing blood vessels that express a reporter protein with the transgenic zebrafish containing blood vessels that express a reporter protein and has an altered blood vessel related gene; and
d) determining the effect of the altered gene on blood vessel growth, such that if blood vessel growth in the transgenic zebrafish containing blood vessels that expresses a reporter protein is less than blood vessel growth in the zebrafish containing blood vessels that express a reporter protein and has an altered blood vessel related gene, the blood vessel related gene is a pro-angiogenic gene that modulates the effects of an anti-angiogenic compound.

17. The method of claim 16, wherein the reporter protein is a fluorescent protein.

18. The method of claim 17, wherein the fluorescent protein is green coral reef fluorescent protein.

19. The method of claim 17, wherein the fluorescent protein is red fluorescent protein.

20. The method of claim 16, wherein the reporter protein is under the control of a VEGFR2 expression sequence.

21. A method of identifying an anti-angiogenic blood vessel related gene that modulates the effects of a pro-angiogenic compound comprising:
a) contacting a transgenic zebrafish containing blood vessels that express a reporter protein with a pro-angiogenic compound;
b) contacting a transgenic zebrafish containing blood vessels that express a reporter protein and has an altered blood vessel related gene with a pro-angiogenic compound;
c) comparing the transgenic zebrafish containing blood vessels that express a reporter protein with the transgenic zebrafish containing blood vessels that express a reporter protein and has an altered blood vessel related gene; and
d) determining the effect of the altered gene on blood vessel growth, such that if blood vessel growth in the transgenic zebrafish containing blood vessels that express a reporter protein is greater than blood vessel growth in the zebrafish containing blood vessels that express a reporter protein and has an altered blood vessel related gene, the blood vessel related gene is an anti-angiogenic gene that modulates the effects of a pro-angiogenic compound.

22. The method of claim 21, wherein the reporter protein is a fluorescent protein.

23. The method of claim 22, wherein the fluorescent protein is green coral reef fluorescent protein.

24. The method of claim 22, wherein the fluorescent protein is red fluorescent protein.

25. The method of claim 21, wherein the reporter protein is under the control of a VEGFR2 expression sequence.

26. A method of identifying a pro-angiogenic compound that modulates the effects of an anti-angiogenic blood vessel related gene:
a) contacting a transgenic zebrafish containing blood vessels that express a reporter protein and has an altered anti-angiogenic blood vessel related gene with a test compound;
b) comparing the transgenic zebrafish containing blood vessels that express a reporter protein and has an altered blood vessel-related gene contacted with the test compound with the blood vessels of a transgenic zebrafish containing blood vessels that express a reporter protein and has an altered blood vessel-related gene that was not contacted with the test compound; and
c) determining the effect of the test compound on blood vessel growth, such that if blood vessel growth in the transgenic zebrafish containing blood vessels that express a reporter protein and has an altered blood vessel related gene contacted with a test compound is greater than blood vessel growth in the zebrafish containing blood vessels that express a reporter protein and has an altered blood vessel related gene, the test compound is a pro-angiogenic compound that modulates the effects of an anti-angiogenic blood vessel related gene.

27. The method of claim 26, wherein the reporter protein is a fluorescent protein.

28. The method of claim 27, wherein the fluorescent protein is green coral reef fluorescent protein.

29. The method of claim 27, wherein the fluorescent protein is red fluorescent protein.

30. The method of claim 26, wherein the reporter protein is under the control of a VEGFR2 expression sequence.

31. A method of identifying an anti-angiogenic compound that modulates the effects of a pro-angiogenic blood vessel related gene:
a) contacting a transgenic zebrafish containing blood vessels that express a reporter protein and has an altered pro-angiogenic blood vessel related gene with a test compound;
b) comparing the transgenic zebrafish containing blood vessels that express a reporter protein and has an altered blood vessel-related gene contacted with the test compound with the blood vessels of a transgenic zebrafish containing blood vessels that express a reporter protein and has an altered blood vessel-related gene that was not contacted with the test compound; and
c) determining the effect of the test compound on blood vessel growth, such that if blood vessel growth in the transgenic zebrafish containing blood vessels that express a reporter protein and has an altered blood vessel related gene contacted with a test compound is less than blood vessel growth in the zebrafish containing blood vessels that express a reporter protein and has an altered blood vessel related gene, the test compound is an anti-angiogenic compound that modulates the effects of a pro-angiogenic blood vessel related gene.

32. The method of claim 31, wherein the reporter protein is a fluorescent protein.

33. The method of claim 32, wherein the fluorescent protein is green coral reef fluorescent protein.

34. The method of claim 32, wherein the fluorescent protein is red fluorescent protein.

35. The method of claim 31, wherein the reporter protein is under the control of a VEGFR2 expression sequence.
